(19) United States
(12) Patent Application Publication
(10) Pub. No.: US 2022/0134370 A1
(43) Pub. Date: May 5, 2022

(54) ELECTROSTATIC SPRAYER NOZZLE

(71) Applicant: Digital Ally, Inc., Lenexa, KS (US)

(72) Inventors: James W. Farnham, IV, Olathe, KS (US); Steven L. Phillips, Olathe, KS (US)

(21) Appl. No.: 17/519,259

(22) Filed: Nov. 4, 2021

Related U.S. Application Data
(60) Provisional application No. 63/109,948, filed on Nov. 5, 2020.

(51) Int. Cl. B05B 14/00; B05B 5/08; B05B 5/035; A61L 2/22; A61L 2/26

(52) U.S. Cl. CPC B05B 14/00; B05B 5/087; B05B 5/035; A61L 2202/121; A61L 2/26; A61L 2202/25; A61L 2202/15; A61L 2/22

(57) ABSTRACT

A system and method for providing an electrostatic sprayer nozzle assembly for dispensing a fluid in a stream of charged particles. The nozzle assembly includes an interior chamber, a nozzle opening on the rear side for receiving a nozzle therethrough, an electrode within the interior chamber for providing a charge to the fluid particles, and a visor connected to an outer surface configured to redirect lost fluid that collects on the outer surface of the assembly back into the fluid stream.

### RELATED APPLICATION

This application claims benefit and priority to U.S. Provisional Patent Application 63/109,948, filed Nov. 5, 2020, entitled ELECTROSTATIC SPRAYER NOZZLE, the disclosure of which is incorporated herein by reference.

### BACKGROUND

### 1. Field

Embodiments of the invention relate to a nozzle design for a liquid sprayer device. More specifically, embodiments of the invention relate to a nozzle design for an electrostatic sprayer device.

### 2. Related Art

Electrostatic spraying has drawn increased interest through recent public health emergencies due to the need to disinfect large indoor spaces (e.g., schools, offices, hospitals, businesses) or areas with many surfaces. Traditionally, these places are cleaned by spraying a fluid disinfectant onto surfaces and wiping down the surface with a cloth. However, such cleaning methods have been shown to be ineffective for preventing the spread of infectious disease.

An improved mechanism for disinfecting surfaces uses an electrostatic delivery spray system. Unlike conventional spraying methods, electrostatic sprayers apply a charge to liquids to be distributed. The charged liquid is attracted to oppositely charged surfaces, which allows for more efficient coating of many surfaces.

However, existing electrostatic delivery systems are inefficient and costly. What is needed is a better process for spreading and applying liquids to surface or locations, such as disinfectants or agricultural products. The current invention applies fluids more efficiently and effectively by minimizing the amount of lost fluid product.

Additionally, a common problem in electrostatic induction sprayers is high voltage leakage current from the electrode. Any leakage current puts additional load on the power supply, reduces efficiency, and can be a safety hazard. In electrostatic induction designs, the fluid must be at ground potential, which is typically achieved by grounding the nozzle tip. Therefore, a very large resistance is required between the electrode and the grounded nozzle tip. Existing designs address this problem of isolation by creating an electrically tortuous, high resistance and high electrical breakdown path between the electrode and ground. This leakage problem is greatly aggravated when the nozzle housing and the electrode becomes moist, which happens in a liquid sprayer. Embodiments of the present invention solve this problem by providing a novel electrode design and composition.

The present invention may be used in a variety of fields, such as health-related fields, agriculture, safety, and law enforcement. Particularly, use is contemplated in vehicles that may be used by multiple participants, such as taxis, vehicles used in ride-share services, law enforcement vehicles, hospital paratransit, and ambulances. Additional uses may include any high-traffic public-use areas, such as hospitals, theaters, amusement parks, office buildings, or public transit systems. The invention may be for commercial or home-use. The inventive electrostatic sprayer nozzle may be used on different sprayer devices of varying sizes and shapes.

The invention describes an improved system for an electrostatic sprayer device and nozzle assembly that minimized the loss of fluid.

### SUMMARY

Embodiments of the invention solve the above-mentioned problems by providing a system and method for using an electrostatic sprayer device and nozzle assembly including a visor for redirecting lost fluid.

A first embodiment of the invention is directed to an electrostatic sprayer nozzle assembly for dispensing a fluid in a stream of charged particles. The nozzle assembly has an outer surface, a top side, a first side, a second side, a rear side, and a front side. The nozzle assembly comprises an interior chamber; a nozzle opening on the rear side for receiving a nozzle therethrough; an electrode within the interior chamber for providing a charge to the fluid particles; and a visor connected to the outer surface configured to redirect lost fluid that collects on the outer surface into the stream.

Another embodiment is directed to an electrostatic sprayer device comprising: a reservoir containing a fluid; a pump for directing the fluid from the reservoir to a nozzle; and a nozzle assembly for dispensing the fluid from the nozzle in a stream of charged fluid particles. The nozzle assembly has an outer surface, a top side, a first side, a second side, a rear side, and a front side. The nozzle assembly comprises an interior chamber; a nozzle opening on the rear side for receiving the nozzle therethrough; an electrode within the interior chamber for providing a charge to the fluid particles; and a visor attached to the outer surface configured to collect and redirect lost fluid into the stream.

Yet other embodiments of the invention are directed to a method for providing a disinfectant to a surface to be cleaned comprising: providing an electrostatic sprayer, providing a positive charge to the fluid particles within a nozzle assembly; spraying the positively charged particles onto a negatively charged surface; and collecting fluid from the outer surface of the nozzle assembly and redirecting the lost fluid into the stream. The sprayer comprises a reservoir containing a disinfectant fluid; a pump for directing the disinfectant fluid from the reservoir to a nozzle; and a nozzle assembly for dispensing the disinfectant fluid from the nozzle in a stream of charged fluid particles, said nozzle assembly having an outer surface, a top side, a first side, a second side, a rear side, and a front side, said nozzle assembly comprising: an interior chamber; a nozzle opening on the rear side for receiving the nozzle therethrough; an electrode within the interior chamber for charging the fluid particles; and a visor attached to the outer surface configured to collect and redirect lost fluid into the stream.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the invention will be apparent from the following detailed description of the embodiments and the accompanying drawing figures.

### BRIEF DESCRIPTION OF THE DRAWING FIGURES

Embodiments of the invention are described in detail below with reference to the attached drawing figures, wherein:

FIG. 1 is a perspective view of a liquid sprayer device including a first embodiment of a sprayer nozzle of the invention;

FIG. 2 is a side view of the sprayer nozzle of the first embodiment of the invention;

FIG. 3 is another side view of the sprayer nozzle of the first embodiment of the invention;

FIG. 4 is a top view of the sprayer nozzle of the first embodiment of the invention;

FIG. 5 is a bottom perspective view of the sprayer nozzle of the first embodiment of the invention;

FIG. 6 is a bottom view of the sprayer nozzle of the first embodiment of the invention;

FIG. 7 is an exploded view of the sprayer nozzle of the first embodiment of the invention;

FIG. 8 is a bottom perspective view of the sprayer nozzle showing a second embodiment of the electrode of the invention;

FIG. 9 is a bottom perspective view of the sprayer nozzle showing a third embodiment of the electrode of the invention; and

FIG. 10 is a bottom perspective view of an embodiment of the sprayer nozzle including a finger guard.

The drawing figures do not limit the invention to the specific embodiments disclosed and described herein. The drawings are not necessarily to scale, emphasis instead being placed upon clearly illustrating the principles of the invention.

### DETAILED DESCRIPTION

The following detailed description references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the invention. The following detailed description is, therefore, not to be taken in a limiting sense. The scope of the invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the technology can include a variety of combinations and/or integrations of the embodiments described herein.

Electrostatic sprayers are based on the principle that opposite charges attract and like charges repel. In one embodiment, by positively charging the fluid particles that are to be sprayed, the fluid particles will be attracted to the negatively charged surfaces. Additionally, the charged particles of fluid will be repelled from each other, thus resulting in a more even distribution of particles over the surface. In some embodiments, the fluid to be sprayed may be a water-based liquid solution.

Exemplary liquids that can be used in the sprayer include disinfectants, cleaners, and antimicrobial fluids, such as bleach. Specifically, the liquid may be a cleaner designed for cleaning and disinfecting hard, non-porous environmental surfaces in health-care facilities. Disinfectants may be those used against viruses and bacteria such as the coronavirus, MRSA, HIV, Listeria, Influenza A, Hepatitis C and Salmonella among many others. Disinfectants may include those listed on the EPA's List N: Disinfectants for Use Against SARS-CoV-2, the virus that causes the novel COVID-19 disease also known as the coronavirus.

Known methods of applying a charge to a liquid in an electrostatic sprayer include direct charging (such as conduction) and indirect charging (such as induction). In a conduction system, a coronal charge is typically applied to the entire reservoir of fluid. This presents safety risks. In an induction system, the charge is not applied to the fluid until after the fluid leaves the nozzle. Thus, the fluid is first atomized and then the particles are charged.

In indirect charging, the fluid is passed through a medium, such as air, that has been electrostatically charged by one or more electrodes to create a static field through which the fluid passes. When the atomized particles of fluid pass through this static field, a charge is imparted on each particle. In some embodiments, a positive charge may be imparted on the atomized particles. In other embodiments, a negative charge may be imparted on the atomized particles. In alternative embodiments, direct charging may be used.

In some embodiments, the fluid is charged by an inductive method. In some embodiments, an electrode is located in an interior chamber of the nozzle assembly. In some embodiments, an electrode is located at or near the nozzle exit to apply a charge to the fluid particles as they exit the nozzle. The charged fluid may be dispersed by the nozzle to form a mist, fog, plume, or spray that can be directed onto a surface.

In some embodiments, the electrostatic sprayer device may be portable and/or battery-powered. In some embodiments, the electrostatic sprayer device may be in the form of a hand-held unit or a backpack.

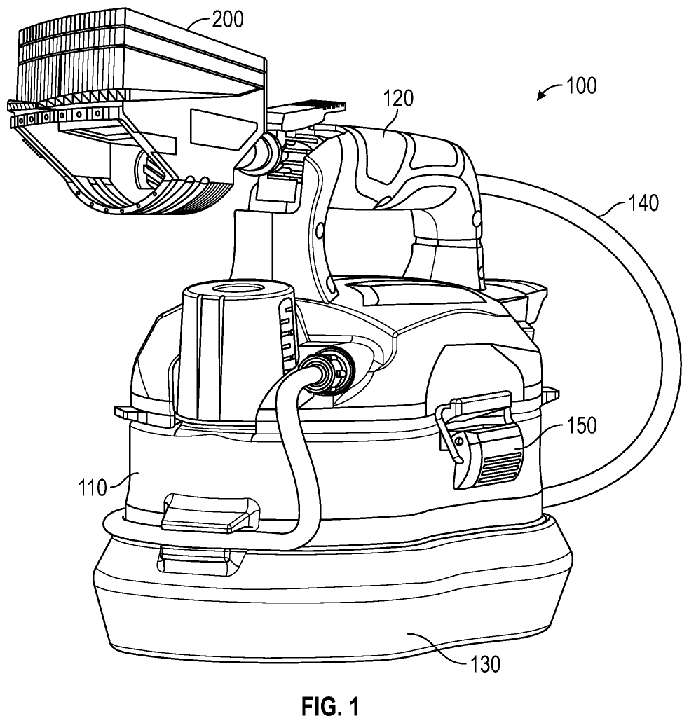

FIG. 1 shows system 100 including a housing 110 having a handle 120 that is sized and shaped to be held by a user. System 100 includes a base 130 having a reservoir therein for holding a fluid. Also contained within the housing 110 is a pump for propelling the fluid through a conduit 140 toward a nozzle assembly 200 for delivery. Housing 110 can be opened to fill the fluid reservoir and includes a closure mechanism 150 for sealing the housing 110 in a closed position.

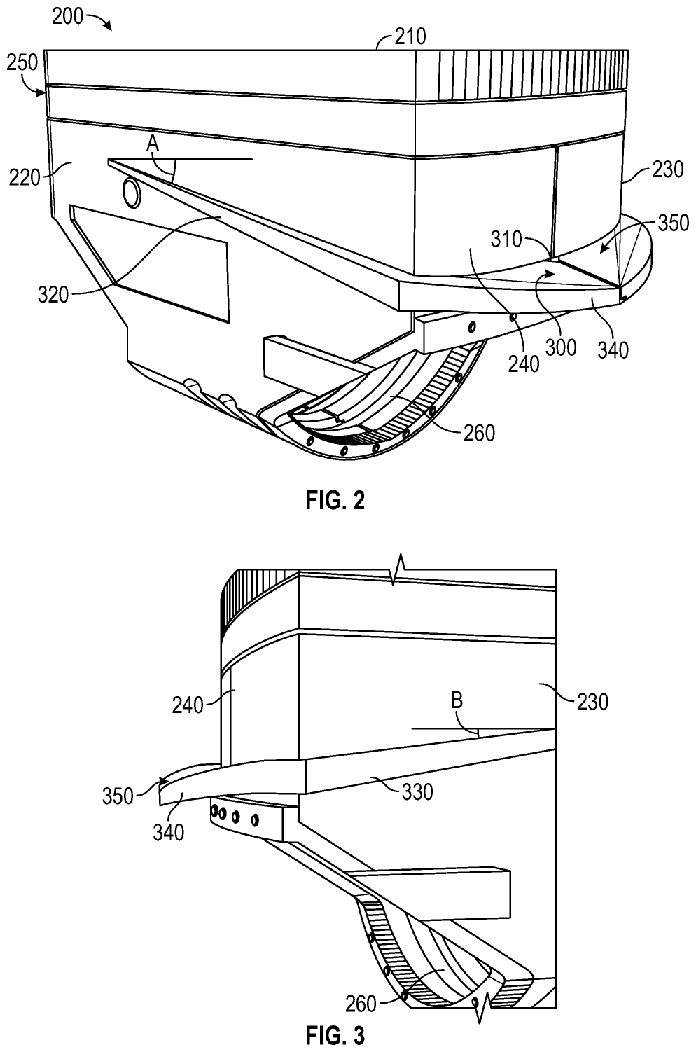

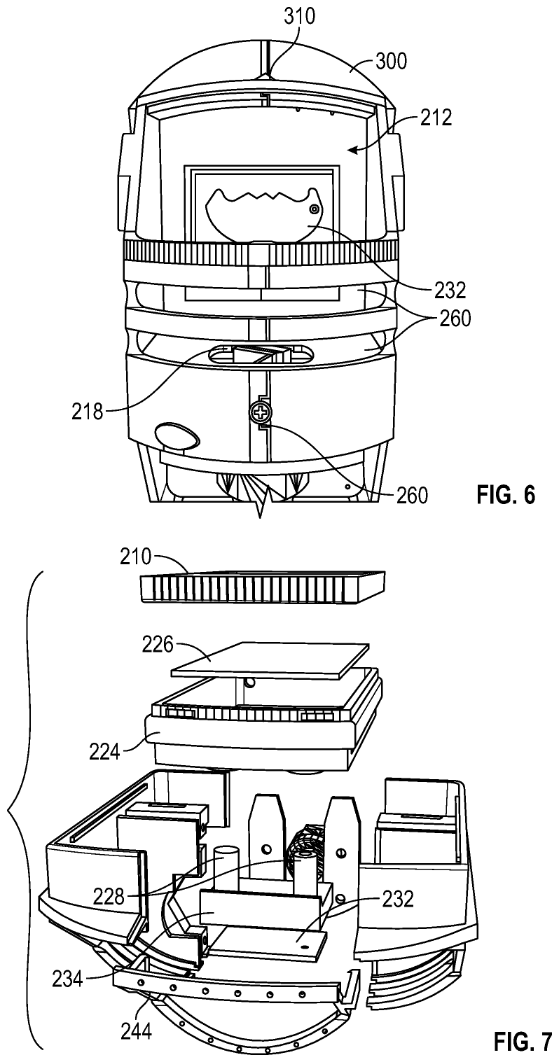

The nozzle assembly is shown in more detail in FIGS. 2-10. As seen in FIGS. 2-3, nozzle assembly 200 has a top side 210, a first side 220, a second side 230, a front side 240, and a rear side 250. A visor 300 may be attached to the outer surface of the nozzle assembly 200 extending along the first side 220, the second side 230, and the front side 240. The visor 300 is provided to collect fluid that can accumulate on the outer surface of the housing during operation and recycle this fluid back into the main fluid stream. The bottom of the nozzle assembly 200 includes additional openings 260 to prevent fluid from collecting therein and to promote air flow therethrough.

Visor 300 includes a first side portion 320 that extends along the first side 220 of the nozzle assembly 200, a second side portion 330 that extends along the second side 230 of the nozzle assembly 200, and a front portion 340 that extends along the front side 240 of the nozzle assembly 200. In some embodiments, first side portion 320 of visor 300 may be sloped downwards towards the front side 240 at an angle A of about 30° to about 60° on the first side 220. In some embodiments, second side portion 330 of visor 300 may be sloped downwards towards the front side 240 at an angle B of about 30° to about 60° on the second side 230. In some embodiments angle A and angle B are equal. In some embodiments, angle A and angle B are not equal. As can be seen in FIG. 2, first side portion 320 extends laterally from the first side portion 320 a distance of about 0.2 inch to about 0.5 inch at its widest part. The first side portion 320 has a tapering width that is widest at the front corner intersecting the front portion 340. Second side portion 330 may be designed to have dimensions similar to that of first side portion 320.

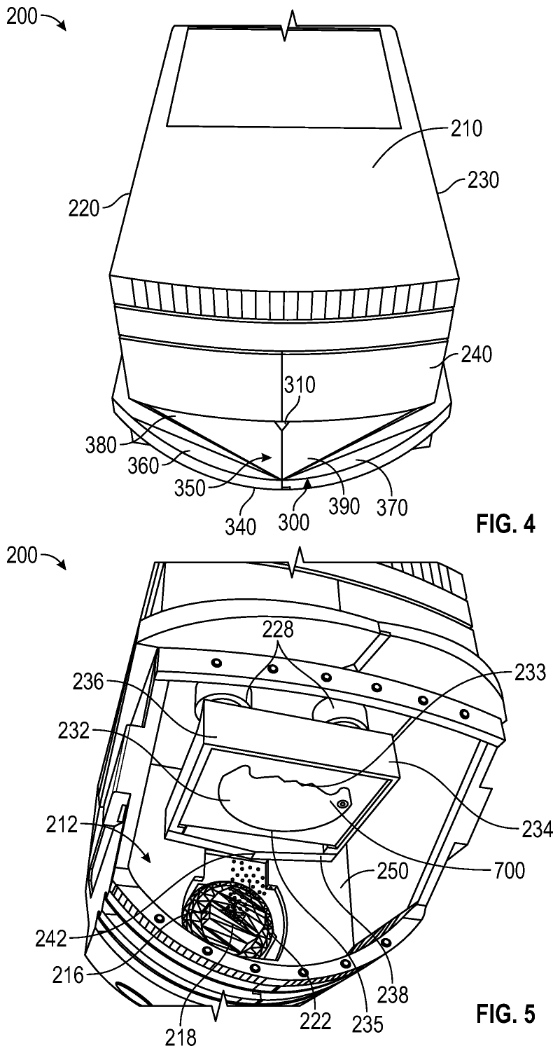

In some embodiments, front portion 340 extends to form a substantially semicircular top surface 350, as seen best in FIG. 4. In some embodiments, the front portion 340 may be otherwise shaped so as to form a curved front edge. In some embodiments, front portion 340 may extend to form a substantially rectangular extension. In some embodiments, front portion 340 includes a small hole 310 in the center of the front side 240. In some embodiments, front portion 340 may include a first sloped fan portion 360, a second sloped fan portion 370, a first trough portion 380, and a second trough portion 390. The portions 360, 370, 380 and 390 are all sloped downwards to cause any fluid to flow towards the central hole 310. In particular, in some embodiments, first and second sloped fan portions 360, 370 may be sloped downwards at an angle C of about 30° to about 60°. in some embodiments, first and second sloped fan portions 360, 370 may be sloped downwards at an angle C of about 45°. In some embodiments, first and second trough portions 380, 390 may be sloped downwards at an angle D of about 30° to about 45°. In some embodiments, first and second trough portions 380, 390 may be sloped downwards at an angle D of about 35°. In some embodiments, angle C is greater than angle D.

The visor 300 is designed to collect fluid that may be located on top side 210, first side 220, second side 230, top surface 350 of the visor, or on a bottom surface of the visor 300. All fluid is directed towards the hole 310 at the center of the trough portions 380, 390 to drip back into the fluid stream.

As can be seen in FIG. 5, nozzle assembly 200 includes an interior chamber 212. Rear side 250 of the nozzle assembly 200 has an opening in the wall for receiving a nozzle 216 therethrough. Nozzle 216 has an outlet 218 for dispensing fluid therefrom. As the fluid exits outlet 218, the fluid is atomized into a plurality of particles 222. Nozzle assembly 200 includes a top portion forming an enclosure 224 for housing a main printed circuit board (PCB) 226. Enclosure 224 is sealed to maintain PCB 226 in a dry state.

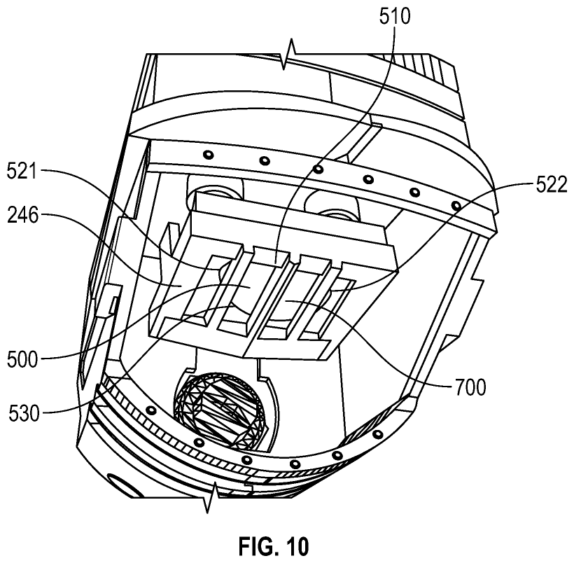

Extending from a lower surface of enclosure 224 is a plurality of supports 228 for supporting an electrode 232 thereon. At least one of the supports 228 is hollow for housing an electrical wire therein to supply power to the electrode 232. This area must remain dry to prevent arcing. The electrode 232 is mounted within a frame 234 that is attached to the support 228. In some embodiments, a finger guard 246 may be provided over the frame 234, as shown in FIG. 10, to prevent a user from touching the electrode.

The electrode frame 234 is mounted within the interior chamber 212 of the nozzle assembly 200 such that the electrode 232 is located about 5-6 mm above the fluid stream. The electrode 232 is also located about 1 inch horizontally forward of the outlet 218 of the nozzle 216. Thus, the fluid will be charged by the electrode 232 after atomization of the particles 222.

As can be seen in FIG. 5, electrode 232 has a non-linear shape configured to optimize the electrostatic field for an exemplary fan shaped spray pattern. Electrode 232 has a substantially semicircular shape, with a wavy front edge 233 having a plurality of peaks and a rear substantially curved edge 235.

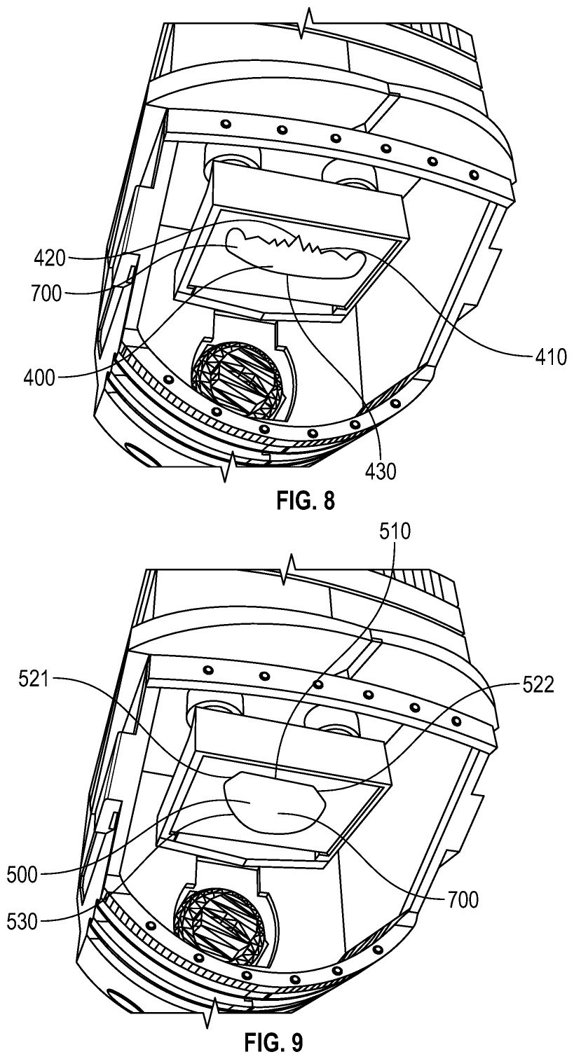

FIGS. 8-9 show additional embodiments of an electrode of the invention. Electrodes 232, 400, and 500 may be coated with a dielectric material 700. The dielectric material 700 opens any possible circuit path between the electrode and ground, which greatly simplifies the mechanical design and guarantees minimal leakage. This dielectric material 700 does not break down at high voltages, provides low leakage at high voltages, and has a low permittivity value in order to not attenuate the electrostatic field. Additionally, the dielectric material 700 increases safety for the operator because it prevents direct contact with the high voltage electrode.

In some embodiments, the dielectric material 700 is also hydrophobic to reduce moisture collection. Moisture collection on the electrode attenuates the electrostatic field and lowers the breakdown voltage around the electrode. The moisture can also increase the leakage issue. In some embodiments, the dielectric material is polytetrafluoroethylene (PTFE). In some embodiments, the dielectric material is PTFE with a silicon backed adhesive.

Once an insulator, such as dielectric coating 700, is placed on the electrode, the electrostatic field is reduced. One way to offset this is to increase the high voltage, however but this increases cost, complexity, and reliability. In some embodiments, the shape of the electrode is optimized to create a maximum field strength and create a field pattern that matches the density of the fluid spray.

FIG. 8 shows a second embodiment of electrode 400, which has a non-linear shape configured to optimize the electrostatic field for a fan shaped spray pattern. Electrode 400 creates a strong field along the jagged front edge 410 with even stronger fields at the center peak 420 of jagged edge 410. Rear edge 430 has a substantially curved shape.

FIG. 9 shows a third embodiment of an electrode of the invention, which has a non-linear shape configured to optimize the electrostatic field for a fan shaped spray pattern. Electrode 500 has a substantially semicircular shape, with a flat front edge 510, two sloped front sides 521, 522 and a rear substantially curved edge 530.

In some embodiments, the electrodes 232, 400, and 500 may be fabricated using printed circuit technology to accommodate a desired intricate shape while keeping the cost low. The printed configuration of electrodes 232, 400, and 500 also makes it simple to solder a high voltage feed to the electrode.

In some embodiments, the nozzle 216 is not electrically grounded directly. Instead, a ground electrode may be placed within the nozzle tube just before the nozzle outlet 218. In some embodiments, a plastic nozzle tip may be used, allowing for lower cost.

In some embodiments, frame 234 is formed as a substantially rectangular box with four vertical walls 236. In some embodiments, an extension 238 is formed on one side of the frame 234. In some embodiments, extension 238 is formed on the rear side of the frame 234 that is closest to the outlet 218. Extension 238 may be formed as a substantially triangular shape which has a lower point 242, as shown in FIG. 5. Extension 238 is configured such that any fluid the collects on frame 234 will be directed towards the lower point 242, such that the fluid will drip back into the fluid stream and be recycled. Although a rectangular shape is shown for frame 234 in FIG. 5, other shapes or sizes for electrode support are contemplated.

In some embodiments, extension 238 and/or visor 300 may be coated with a layer of hydrophobic material. In some embodiments, part or all of extension 238 and/or visor 300 may be composed of a hydrophobic material to facilitate the collection of the fluid. A hydrophobic material will prevent fluid from collecting on the extension 238 and/or visor 300 and fluid droplets will be repelled therefrom. An exemplary hydrophobic material or coating may be polytetrafluoroethylene (PTFE).

In some embodiments, a safety guard 244 may be applied to the front of the nozzle assembly 200 opening to prevent a user from inserting their hands or other objects inside. In some embodiments, the guard 244 may include a plurality of monofilaments that are attached to a front surface of the nozzle assembly 200. In some embodiments, the monofilaments may be attached to the front surface to create vertical lines.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. An electrostatic sprayer nozzle assembly for dispensing a fluid in a stream of charged particles, said nozzle assembly having an outer surface, a top side, a first side, a second side, a rear side, and a front side, said nozzle assembly comprising:
an interior chamber;
a nozzle opening on the rear side for receiving a nozzle therethrough;
an electrode within the interior chamber for providing a charge to the fluid particles; and
a visor connected to the outer surface configured to redirect lost fluid that collects on the outer surface into the stream.

2. The electrostatic sprayer nozzle assembly of claim 1, wherein the visor comprises a front portion extending forward from the front side of the nozzle assembly.

3. The electrostatic sprayer nozzle assembly of claim 2, wherein the front portion comprises a central hole.

4. The electrostatic sprayer nozzle assembly of claim 3, wherein the visor is sloped to direct the fluid towards the central hole.

5. The electrostatic sprayer nozzle assembly of claim 1, wherein the visor comprises a first side portion extending along the first side of the nozzle assembly and a second side portion extending along the second side of the nozzle assembly.

6. The electrostatic sprayer nozzle assembly of claim 1, wherein the visor comprises a hydrophobic coating on at least one surface.

7. The electrostatic sprayer nozzle assembly of claim 1, further comprising an electrode frame supporting the electrode, wherein the electrode frame comprises a lower extension configured to redirect fluid that collects on the electrode frame into the stream.

8. The electrostatic sprayer nozzle assembly of claim 7, wherein the lower extension comprises a hydrophobic coating on at least one surface.

9. An electrostatic sprayer device comprising:
a reservoir containing a fluid;
a pump for directing the fluid from the reservoir to a nozzle; and
a nozzle assembly for dispensing the fluid from the nozzle in a stream of charged fluid particles,
said nozzle assembly having an outer surface, a top side, a first side, a second side, a rear side, and a front side, said nozzle assembly comprising:
an interior chamber;
a nozzle opening on the rear side for receiving the nozzle therethrough;
an electrode within the interior chamber for providing a charge to the fluid particles; and
a visor attached to the outer surface configured to collect and redirect lost fluid into the stream.

10. The electrostatic sprayer device of claim 9, wherein the visor comprises a front portion extending forward from the front side of the nozzle assembly.

11. The electrostatic sprayer device of claim 10, wherein the front portion has a central hole and the visor is sloped to direct the fluid towards the central hole.

12. The electrostatic sprayer device of claim 9, further comprising a hydrophobic coating on at least one surface.

13. The electrostatic sprayer device of claim 9, wherein the electrode comprises a wavy front edge having a plurality of peaks.

14. The electrostatic sprayer device of claim 9, further comprising an electrode frame supporting the electrode, wherein the electrode frame comprises a lower extension configured to redirect fluid that collects on the electrode frame into the stream.

15. The electrostatic sprayer device of claim 14, further comprising a safety guard attached to the front side.

16. A method for providing a disinfectant to a surface to be cleaned comprising:
providing an electrostatic sprayer, said sprayer comprising:
a reservoir containing a disinfectant fluid;
a pump for directing the disinfectant fluid from the reservoir to a nozzle; and
a nozzle assembly for dispensing the disinfectant fluid from the nozzle in a stream of charged fluid particles,
said nozzle assembly having an outer surface, a top side, a first side, a second side, a rear side, and a front side, said nozzle assembly comprising:
an interior chamber;
a nozzle opening on the rear side for receiving the nozzle therethrough;
an electrode within the interior chamber for charging the fluid particles; and
a visor attached to the outer surface configured to collect and redirect lost fluid into the stream;
providing a positive charge to the fluid particles within the nozzle assembly;
spraying the positively charged particles onto a negatively charged surface; and
collecting fluid from the outer surface of the nozzle assembly and redirecting the lost fluid into the stream.

17. The method of claim 16, wherein the visor comprises a sloped front portion extending forward from the front side of the nozzle assembly.

18. The method of claim 17, wherein the front portion comprises a central hole, the method further comprising: directing the fluid towards the central hole.

19. The method of claim 16, further comprising: providing a positive charge to the fluid particles via the electrode.

20. The method of claim 16, further comprising: providing a hydrophobic coating on at least one surface.
